(19) United States
(12) Patent Application Publication
(10) Pub. No.: US 2014/0371786 A1
(43) Pub. Date: Dec. 18, 2014

(54) EXPANDABLE MEDICAL ANCHOR DEVICE FORMED OF CUT METAL TUBE

(71) Applicant: Calore Medical Ltd., Or Akiva (IL)

(72) Inventors: Giora Kornblau, Binyamina (IL); David Maier Neustadter, Nof Ayalon (IL)

(21) Appl. No.: 14/472,912

(22) Filed: Aug. 29, 2014

Related U.S. Application Data
(63) Continuation-in-part of application No. 14/250,716, filed on Apr. 11, 2014.
(60) Provisional application No. 61/811,743, filed on Apr. 14, 2013.
(60) Provisional application No. 61/872,722, filed on Sep. 1, 2013.
(60) Provisional application No. 61/894,445, filed on Oct. 23, 2013.
(60) Provisional application No. 62/015,968, filed on Jun. 23, 2014.

(51) Int. Cl. A61B 17/08

(52) U.S. Cl. CPC A61B 17/08
USPC 606/213

(57) ABSTRACT

A radially expandable anchor device formed of a cut metal tube is described. The tubular wall of the cut metal tube has a plurality of slits thereon, which together form a plurality of bands. The plurality of slits is configured to allow the plurality of bands to splay outward in the form of overlapping petals when an axial compressive force is applied to the cut metal tube. The cut metal tube may have a combination of radial and non-radial cuts. The non-radial cuts allow the adjacent sections of the tubular wall on opposing sides of the cut to overlap each other when they are pressed together by an axial compressive force, thus facilitating the radial expansion of the cut metal tube.

### CROSS-REFERENCES TO RELATED APPLICATIONS

This application is a continuation-in-part of U.S. Non-Provisional application Ser. No. 14/250,716, filed Apr. 11, 2014, which claims priority to U.S. Provisional Application No. 61/811,743, filed Apr. 14, 2013. This application also claims priority to U.S. Provisional Application No. 61/872,722, filed Sep. 1, 2013, U.S. Provisional Application No. 61/894,445, filed Oct. 23, 2013, and U.S. Provisional Application No. 62/015,968, filed Jun. 23, 2014, all of which are incorporated herein by reference in their entirety.

### TECHNICAL FIELD

The present disclosure relates generally to an expandable anchor device for biomedical applications, and more specifically, to a radially expandable anchor device formed of a cut metal tube.

### BACKGROUND

Many expanding medical devices, such as stents, anchors, occluders, filters, baskets, artery locators, etc. can be inserted into the body in a collapsed configuration and expanded once inside the body. Many of these devices are made of metal wire or cut metal tubes. However, different expanding medical devices expand in different ways depending on their intended use. For example, some devices, such as stents, expand by changing only the diameter of the tube, while other devices, such as filters, anchors, artery locators, and occluders, change their entire geometry as they expand.

When attempting to change a narrow tube into a flattened disc, for example, in anchors, artery locators, occluders, etc., mechanical limitations restrict the use of cut metal tubes and favor the use of metal wires. Metal wires can be arranged to lie flat when expanded, forming a geometry similar to the overlapping petals of a flower, as described in U.S. Pat. No. 8,366,706. Such a geometrical transformation may not be easily be achieved by a cut metal tube.

A number of configurations of expandable cut metal tubes, which form metal loops when expanded, have been previously described. For example, U.S. Pat. No. 8,568,445 describes spiral cuts in the wall of a tube, which form struts that expand into loops or petals when compressed. U.S. Pat. No. 8,252,022 describes lengthwise or spiral cuts in a tube, which form expansion members that expand transversely when compressed longitudinally. However, as highlighted by the figures of the expanded form in each of the descriptions, they do not typically form loops that overlap each other like the overlapping petals of a flower.

An overlapping petal pattern may produce a disc-shaped structure that, when covered by an elastic membrane, provides better mechanical support, sturdiness, and sealing performance than non-overlapping loops or petals. The overlapping petal pattern may also provide a large diameter disc relative to the overall length of the device when in its collapsed configuration. For example, in the case of intravascular anchors or artery locators, the ratio of collapsed length to expanded diameter may be particularly important. An intravascular anchor or artery locator is generally inserted into the blood vessel, expanded, pulled gently against the inner wall of the vessel, held in place during the closure procedure, and then re-collapsed and removed. In such applications, the anchor has to be sturdy and have a sufficient expanded diameter size to remain firmly within the vessel, yet be short enough when collapsed so that it does not injure the vessel wall upon re-collapse at the end of the procedure when it is oriented sideways across the diameter of the vessel. Anchors with overlapping petal patterns generally provide these beneficial characteristics. However, wire-based anchors having overlapping petal patterns are expensive to manufacture. Therefore, if an equivalent geometrical configuration could be achieved by using a cut metal tube, the beneficial features of wire-based anchors could be achieved while significantly reducing manufacturing costs.

### SUMMARY

The present disclosure is directed to the design and configuration of an expandable anchor device formed of a cut or slotted metal tube. When an axial compressive force is applied to an exemplary cut metal tube of the present disclosure, the tube expands radially such that the bands between the cuts of the tube bend in such a way that they form an overlapping flower petal pattern.

Some aspects of the present disclosure include an expandable anchor device formed of a cut metal tube for temporarily occluding an opening in a tissue wall while a treatment applicator is used to close or heal the opening. The anchor device of the present disclosure may be used as a component of a device to thermally close puncture sites (i.e., arteriotomies) on blood vessel walls. The anchor device of the present disclosure is not limited to blood vasculature applications, and may be applied to any vessel, duct, canal, tubular structure, and/or cavity in the body. It is to be understood that the term “body canal” in this disclosure refers to any blood vessel, duct, canal, tubular structure, and/or tissue tract within the body.

One embodiment of the present disclosure may include an anchor device comprising an expandable tube, the expandable tube having a first end region and a second end region. The expandable tube may be configured to traverse a perforation in a tissue wall of a body canal and to fit within an interior of the body canal proximate to the perforation. The expandable tube may also include a plurality of primary slits therein, each primary slit extending from the first end region to the second end region, the primary slits cooperating to define a plurality of bands. Each primary slit may comprise at least one substantially longitudinal cut portion and at least one substantially non-longitudinal cut portion extending from the at least one substantially longitudinal cut portion. The primary slits may be configured such that when the first end region and the second end region are compressed towards each other, the plurality of bands splay outward. In another embodiment, the primary slits may be configured such that when the first end region and the second end region are compressed towards each other, the plurality of bands splay outward to form an overlapping petal pattern. In another embodiment, each primary slit may be connected to a serpentine cut on at least one of the first end region and the second end region. In yet another embodiment, the plurality of bands may include at least one secondary slit within each band, the at least one secondary slit having a length shorter than a length of the primary slit.

Another embodiment of the present disclosure may include an anchor device comprising an expandable tube, wherein the expandable tube may be configured to traverse a perforation in a tissue wall of a body canal and to fit within an interior of the body canal proximate to the perforation. The expandable tube includes an elongated central axis and a tubular wall configured to expand radially upon application of an axial compression force along a direction of the central axis. The expandable tube may also comprise at least one non-radial cut in the tubular wall, such that application of the axial compression force results in relative radial motion of surfaces on opposite sides of the cut.

Yet another embodiment of the present disclosure may include an anchor device comprising an expandable tube, wherein the expandable tube may be configured to traverse a perforation in a tissue wall of a body canal and to fit within an interior of the body canal proximate to the perforation. The expandable tube includes a tubular wall configured to expand radially upon application of an axial compression force. The expandable tube may also comprise at least one cut in a non-radial plane through the tubular wall such that adjacent surfaces on opposite sides of the cut are ramped with respect to each other, and wherein application of the axial compression force to the cut tube results in relative radial motion of the surfaces on opposite sides of the cut.

Other embodiments of this disclosure are contained in the accompanying drawings, description, and claims. Thus, this summary is exemplary only, and is not to be considered restrictive.

### BRIEF DESCRIPTION OF DRAWINGS

The accompanying drawings, which are incorporated in and constitute a part of this specification, illustrate the disclosed embodiments and together with the description, serve to explain the principles of the various aspects of the disclosed embodiments. In the drawings:

FIG. 1A shows an exemplary cut metal tube in a compressed state indicating the dimensions ‘l’ and ‘d’, the length and diameter, respectively, of the tube in its compressed state;

FIG. 1B shows the exemplary cut metal tube of FIG. 1A in an expanded state indicating the dimension ‘D’, the diameter of the tube in its expanded state;

FIG. 2 is a cross-sectional view of an exemplary cut metal tube indicating components of the device and the angle between them;

FIG. 3A shows a cut metal tube in a compressed state, in accordance with exemplary embodiments of the present disclosure;

FIG. 3B shows the cut metal tube of FIG. 3A in an expanded state, forming an overlapping flower petal like configuration, in accordance with exemplary embodiments of the present disclosure;

FIG. 4A shows a cut pattern that may be used in a cut metal tube, in accordance with exemplary embodiments of the present disclosure;

FIG. 4B shows how the cut pattern shown in FIG. 4A allows deformation of the strips or bands formed by the cuts, in accordance with exemplary embodiments of the present disclosure;

FIG. 5A shows an expandable cut metal tube in accordance with exemplary embodiments of the present disclosure;

FIG. 5B shows the cut pattern of the expandable cut metal tube shown in FIG. 5A;

FIG. 5C shows another version the cut pattern of the expandable cut metal tube shown in FIG. 5A; and

FIG. 6 shows a cross-sectional view of a cut metal tube with radial and non-radial cuts, in accordance with exemplary embodiments of the present disclosure.

It is to be understood that both the foregoing general description and the following detailed description are exemplary and explanatory only and are not restrictive of the disclosed embodiments, as claimed.

### DESCRIPTION OF EXEMPLARY EMBODIMENTS

Reference will now be made to certain embodiments consistent with the present disclosure, examples of which are illustrated in the accompanying drawings. Wherever possible, the same reference numbers are used throughout the drawings to refer to the same or like parts.

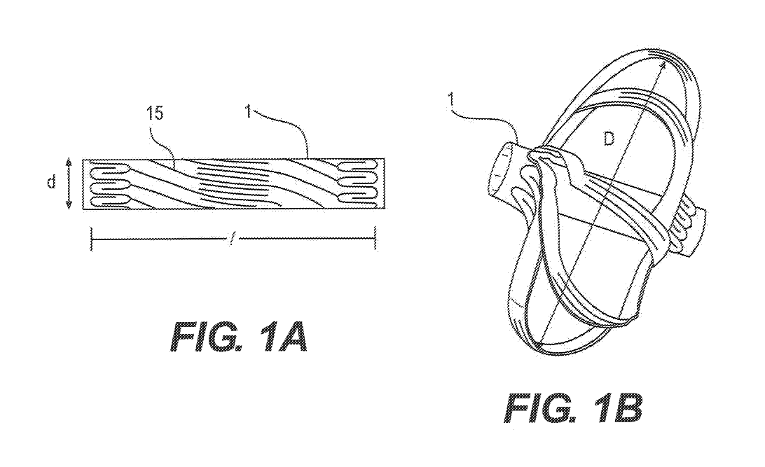

The present disclosure describes an anchor device configured to traverse a perforation or an opening in a tissue wall of a body canal and to fit within an interior space of the body canal proximate to the perforation. Exemplary embodiments of the anchor device are formed of an expandable metal tube having a plurality of slits, slots, cuts, or incisions therein. Such an expandable metal tube is referred to herein as “cut metal tube” or “slotted metal tube.” FIG. 1A shows an exemplary cut metal tube 1 with a plurality of slits 15. In a compressed state, tube 1 has an outer diameter d and a length of the cut region l, as shown in FIG. 1A. Upon application of an axial compressive force, tube 1 expands radially to an expanded diameter D, as shown in FIG. 1B. In exemplary embodiments, the relationship between the compressed length l, the compressed diameter d, and the expanded diameter D are such that l−(D−d) may be less than 25 times the wall thickness of tube 1. In one such exemplary embodiment, tube 1 has a compressed length l of less than 7 mm, a compressed diameter of less than 1.5 mm, and an expanded diameter of greater than 4.5 mm.

In exemplary embodiments, when an axial compressive force is applied to one or both ends of cut metal tube 1, the expandable section of the tube, which comprises the cuts or the silts, expands radially. In some embodiments, the tube may expand radially up to its maximum expanded diameter D. When the axial compressive force is removed, the expandable section of the tube may return to 90% to 150% of its compressed diameter d. In some embodiments, the dimensions of cut metal tube 1 is such that the sum of the compressed length l and the compressed diameter d minus the maximum expanded diameter D is less than 35 times the wall thickness of cut metal tube 1.

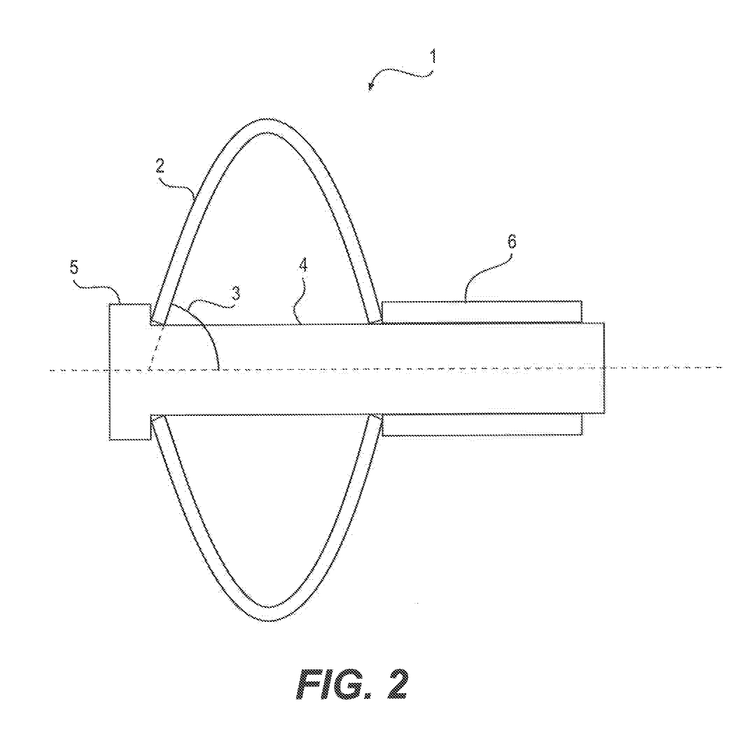

In some embodiments of the present disclosure, the anchor device may comprise a cut metal tube having metal bands between the cuts or the slits. FIG. 2 shows a cross-sectional view of cut metal tube 1 in an expanded configuration. As shown in FIG. 2, tube 1 comprises metal bands 2, which may bend to form an angle 3 with the longitudinal axis of the tube at its ends. In the present disclosure, the term “longitudinal axis of the tube” is used to mean a direction that is within 20 degrees of the direction of the line connecting the center points of the ends of a cut metal tube.

In some embodiments, when bands 2 are in an expanded state, bands 2 form an angle 3 with the longitudinal axis of cut metal tube 1. In one such embodiment, angle 3 may be between 45° and 135°. In another embodiment, angle 3 may be between 60° and 120°. In yet another embodiment, angle 3 may be between 70° and 100°.

In exemplary embodiments, the widths of some locations along the lengths of bands 2 and cut patterns provided at the ends of cut metal tube 1 are configured to induce tube 1 to preferentially bend in a predefined way when compressive force is applied to the ends of tube 1 along the longitudinal axis of the tube. In some embodiments, the widths of some locations along the lengths of bands 2 and cut patterns provided at the ends of cut metal tube 1 are configured to allow the device to bend sufficiently to achieve its intended expanded shape without breaking. In some embodiments, the widths of some locations along the lengths of bands 2 and cut patterns provided at the ends of cut metal tube 1 are configured to allow the device to bend sufficiently to achieve its intended expanded shape while all deformations remain substantially elastic.

In exemplary embodiments, cut metal tube 1 is not connected to the other components of the anchor device of which it forms a part, but rests upon a support element 4, as shown in FIG. 2, which is composed of a wire or tube that fits through cut metal tube 1. In some embodiments, support element 4, upon which cut metal tube 1 rests, has at its distal end a section 5 which has a diameter larger than the inner diameter of cut metal tube 1 in its compressed configuration, such that when support element 5 is pulled in the proximal direction through cut metal tube 1, it applies a force on the distal end of cut metal tube 1 in the proximal direction along the longitudinal axis of the tube.

In some embodiments, the anchor device may comprise a sleeve 6 around the support element 4, proximal to cut metal tube 1, as shown in FIG. 2. Sleeve 6 may have an outer diameter larger than the inner diameter of cut metal tube 1 in its compressed configuration, such that when support element 5 is pulled in the proximal direction, sleeve 6 applies a force on the proximal end of cut metal tube 1 in the distal direction along the longitudinal axis of the tube.

In some embodiments, the deformations that occur during expansion of a cut metal tube of an anchor device are substantially plastic in nature, and therefore, the cut metal tube remains substantially in its expanded configuration even after the expanding forces are removed. In other embodiments, the deformations that occur during expansion of the cut metal tube are substantially elastic in nature, and therefore, the cut metal tube returns substantially to its collapsed tubular configuration when the expanding forces are removed.

In some embodiments, the slots or cuts in the cut metal tube, which define the expandable bands, are substantially parallel with the longitudinal axis of the tube. Further, in some embodiments, the cut metal tube is designed such that when expanded by applying a force along the longitudinal axis of the tube, the bands between the slots of the tube remain substantially parallel to the longitudinal axis of the tube, and bend primarily in the radial direction to expand radially outwards as the ends of the tube come closer together.

Some embodiments include a cut metal tube designed such that when expanded by applying a force along the longitudinal axis of the tube, the bands between the slots of the device twist such that their bending is in both the radial and circumferential directions, forming an overlapping flower petal pattern as the ends of the tube come closer together.

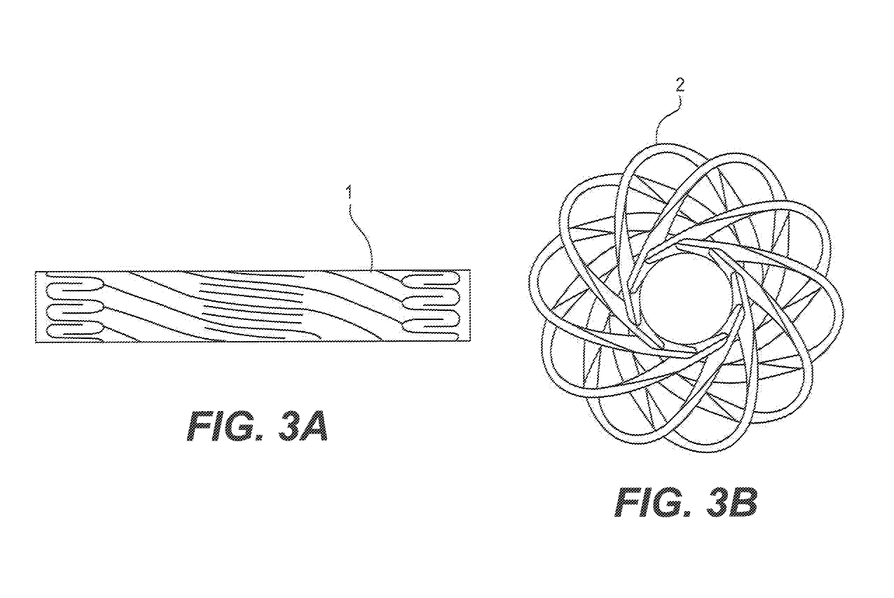

FIG. 3A shows cut metal tube 1 according to one embodiment of the present disclosure, and FIG. 3B shows an overlapping petal pattern formed by bands 2 of cut metal tube 1 in its expanded configuration. In some embodiments, as shown in FIG. 3A, the cuts on cut metal tube 1 may include multiple sections, each with a different angle relative to the longitudinal axis of the tube. In some embodiments, the proximal, distal, and middle sections may contain cuts that are substantially parallel with the longitudinal axis of the tube, but shifted around the tube relative to each other. The portions of the cuts that connect the proximal and middle sections, and those that connect the middle and distal sections, may be angled relative to the longitudinal axis of the tube. Cut patterns may include multiple sections with different angles relative to the longitudinal axis of the tube so as to produce bands 2 with mechanical properties and behavior superior to those obtained by previously described parallel cuts, or spiral cut patterns.

In some embodiments in which the cut metal tube expands to form an overlapping petal pattern, the thickness of the bands and the thickness of the cut patterns in the proximal and distal sections of the tube are such that the twisting that allows the bands to bend in the circumferential direction may take place primarily in the bands, and not in the region of the cut patterns in the proximal and distal sections of the tube.

In some embodiments in which the cut metal tube expands to form an overlapping petal pattern, the thickness of the bands and the thickness of the cut patterns in the proximal and distal sections of the tube are such that the twisting that allows the bands to bend in the circumferential direction may take place primarily in the region of the cut patterns in the proximal and distal sections of the tube, and not in the bands themselves.

Exemplary embodiments of the present disclosure may comprise flexibility enhancing cut patterns in a proximal and/or distal section of a cut metal tube. In the present disclosure, a “flexibility enhancing cut pattern” is defined as a cut pattern that results in strips that include multiple turns, such that the path length of each strip in the region of the flexibility-enhancing cut pattern is significantly longer than the longitudinal length of the region of the cut pattern along the tube.

Some embodiments include an intermediate axial section having a cut pattern that results in substantially parallel strips (i.e., bands) that are oriented substantially along the length of the tube. In such embodiments, the parallel strips of the intermediate section are connected to the strips of the flexibility enhancing cut patterns in the proximal and/or distal sections of the tube.

In some embodiments, the cut patterns of the intermediate axial section result in substantially parallel strips that run substantially parallel to the longitudinal axis of the metal tube. In other embodiments, the cut patterns of the intermediate axial section result in substantially parallel strips that are oriented with a fixed angle relative to the longitudinal axis of the tube, such that the cut patterns twist around the surface of the tube. In some such embodiments, the orientation angle of the strips or bands of the intermediate axial section is such that the strips twist between 90° and 270° C. around the surface of the tube over the length of the intermediate section.

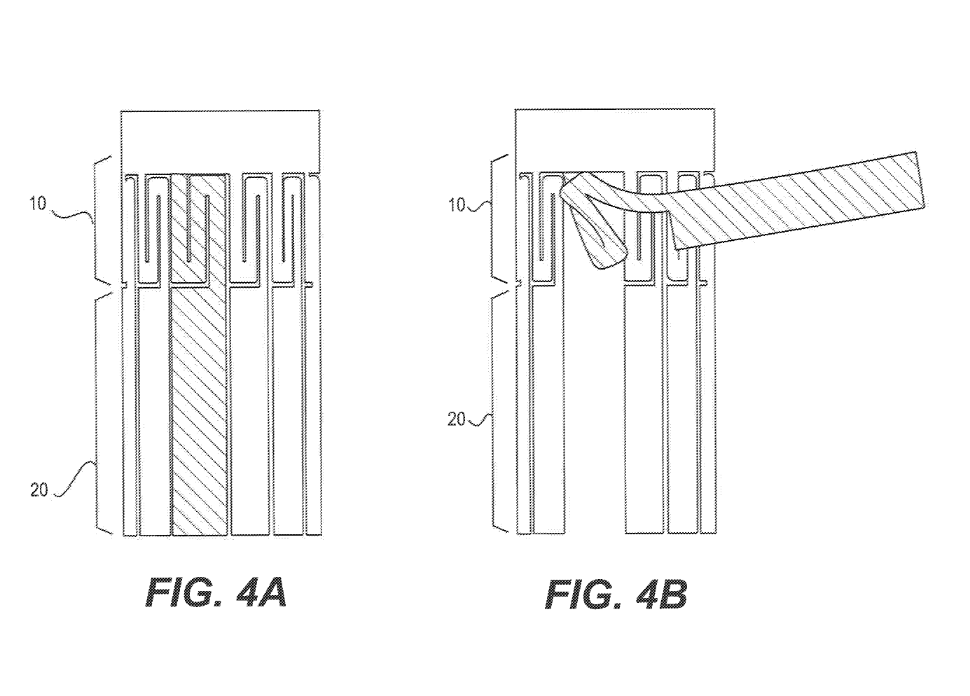

FIGS. 4A and 4B illustrate an exemplary cut pattern that may be used in a cut metal tube. The exemplary cut pattern includes a first axial section 10 having a flexibility enhancing cut pattern and a second axial section 20 having a straight-cut pattern. As shown in FIG. 4A, the strips of sections 10 and 20 may be connected to each other. The flexibility enhancing cut pattern may include cuts that are straight or curved, symmetrical or asymmetrical, and at any orientation. The flexibility enhancing cuts may or may not include empty spaces. In one exemplary embodiment, shown in FIGS. 4A and 4B, the flexibility-enhancing cut pattern has an “S” shape or a doubleback pattern. Sections 10 and 20 may be configured to cooperate with each other, such that when axial compressive force is applied to the cut metal tube, the strips in section 10 may deform to allow the section 20 strips connected thereto to rotate.

In some embodiments, sections 10 and 20 are configured to cooperate with each other such that when the tube is subjected to axial compression force, section 20 expands radially to a substantially greater degree than section 10. FIG. 4B illustrates that the strips of flexibility enhancing cut pattern in section 10 may be deformed to allow a straight-cut strip in axial section 20 to rotate substantially into the axial plane.

In some embodiments, the cut metal tube may include a third axial section having a cut pattern. In some embodiments, second axial section 20 is placed in between first axial section 10 and the third axial section. In one such embodiment, the cut pattern in the third axial section is a flexibility enhancing cut pattern. First axial section 10, second axial section 20, and the third axial section may be configured to cooperate with each other such that when the tube is subjected to an axial compression force, the strips in second axial section 20 expand radially. In some embodiments, when the strips of second axial section 20 expand radially, the total length of first axial section 10, second axial section 20, and the third axial section shortens by more than 90% of the compressed length of second axial section 20. In exemplary embodiments, second axial section 20 expands radially to form an overlapping petal design.

In some exemplary embodiments, as shown in FIGS. 4A and 4B, the cut pattern in second axial section 20 results in substantially parallel strips oriented substantially along the length of the tube. In other exemplary embodiments, the cut pattern in second axial section 20 results in substantially parallel strips oriented with a fixed angle relative to the longitudinal axis of the tube, such that they twist around the surface of the tube. In one such exemplary embodiment, the strips in second axial section 20 twist between 90° and 270° around the surface of the tube over the length of axial section 20.

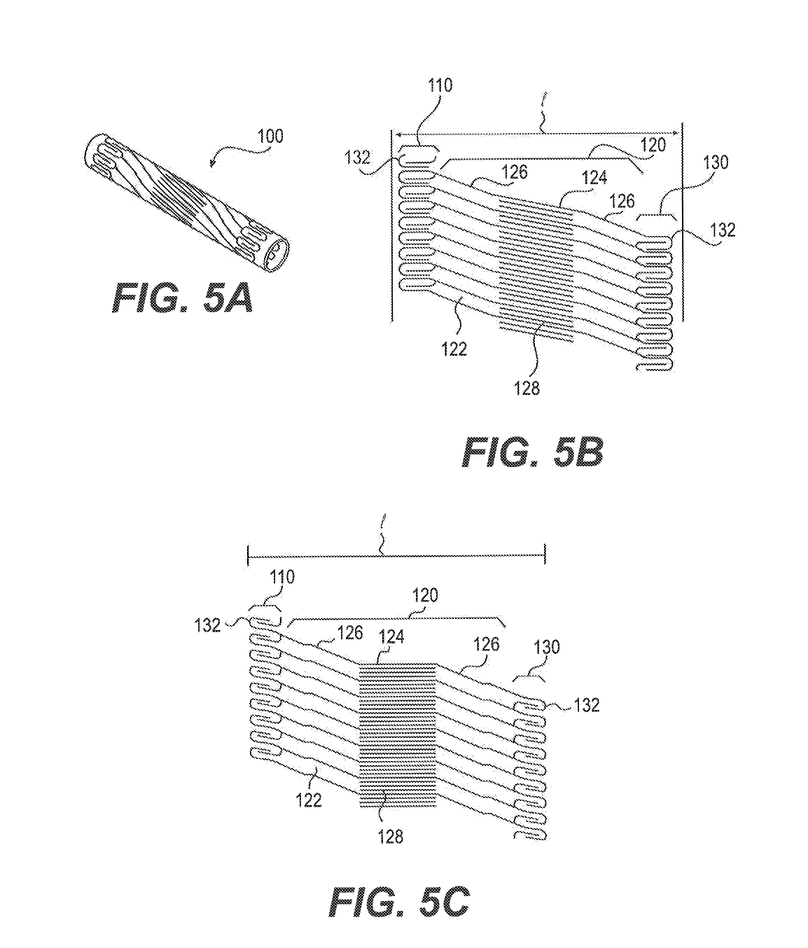

FIG. 5A shows a cut metal tube 100 having an expandable section and cut patterns that enable the cut metal 100 to expand radially when axial compressive force is applied to the tube. An anchor device formed with cut metal tube 100 may be configured to traverse through a tissue perforation or an opening in its compressed state. When cut metal tube 100 is inside a body canal (where the anchor is to be deployed), cut metal tube 100 may be expanded radially to take the form of a flattened disk. In exemplary embodiments, cut metal tube 100 may form an overlapping petal design when expanded. Cut metal tube 100 in its expanded configuration may be positioned in close proximity to the perforation or opening in the tissue wall of the body canal. Following the application of a treatment procedure to the opening in the tissue wall, cut metal tube 100 may be undeployed, i.e., cut metal tube 100 may be returned to its compressed state by removing the axial compression force. Cut metal tube 100 may be retracted in its compressed state from inside the body canal through the opening in the tissue wall, which may be partially closed as a result of the treatment procedure.

FIGS. 5B and 5C illustrate the cut patterns provided on the tubular wall of cut metal tube 100. The cut patterns on cut metal tube 100 are provide to facilitate radial expansion of cut metal tube 100 when an axial compressive force is applied. In exemplary embodiments, the cut pattern is configured to allow expansion of cut metal tube 100 into an overlapping petal pattern.

In exemplary embodiments, as illustrated in FIGS. 5B and 5C, cut metal tube 100 may include a first end region 110, a second end region 130, and an intermediate section 120 extending between first end region 100 and second end region 130. End regions 110 and 130 may comprise flexibility-enhancing cut patterns that may form a plurality of strips that are connected to the strips or bands formed in intermediate section 120. In exemplary embodiments, first end region 100 and/or second end region 130 may include serpentine cuts 132. In one such embodiment, each serpentine cut forms an s-shape. In exemplary embodiments, intermediate section 120 comprises a plurality of primary slits therein, which cooperate to define a plurality of bands 122. The primary slits are interconnected with serpentine cuts 132. In exemplary embodiments, the primary slits are configured such that when first end region 110 and second end region 130 are compressed towards each other, plurality of bands 122 splay outward.

In exemplary embodiments, each primary slit in intermediate section 120 comprises at least one substantially longitudinal cut portion 124 and one or more substantially non-longitudinal cut portions 126 extending from substantially longitudinal cut portion 124. In such embodiments, at least one substantially longitudinal cut portion 124 and one or more substantially non-longitudinal cut portions 126 may together form the primary slit in cut metal tube 100. Further, in such embodiments, at least one substantially longitudinal cut portion 124 and one or more substantially non-longitudinal cut portions 126 may cause the primary slit to have a stepped appearance. In exemplary embodiments, the primary slit may have a substantially constant pitch throughout its length.

In some embodiments, each plurality of bands 122 may further include at least one secondary slit 128 therein. Each secondary slit 128 may have a length shorter than the primary slit. Secondary slits 128 may be configured to facilitate radial expansion of intermediate axial section 120. In some embodiments, the primary slits and secondary slits 128 may be configured such that when a compressive force along the longitudinal axis of cut metal tube 100 is applied, plurality of bands 122 twist such that they bend is in both the radial and circumferential directions forming an overlapping flower petal pattern as the ends of the tube come closer together.

In exemplary embodiments, one or more substantially non-longitudinal cut portions 126 extend 180° around cut metal tube 100. In some such embodiments, each primary slit may comprise a first non-longitudinal slit 126 extending 90° around the tube, followed by a straight slit 124 extending substantially along the longitudinal axis of the tube, and then a second non-longitudinal slit 126 extending 90° around the tube. In such embodiments, the primary slit wraps 180° around cut metal tube 100. The length l of the cut portion of cut metal tube 100, i.e., the total length of first end region 110, intermediate section 120, and second end region 130, may be the appropriate length to form a flattened disk of expanded diameter D when axial compression force is applied to cut metal tube 100.

In exemplary embodiments, cut metal tube 100 may be made of a super elastic material, for example, Nitinol, so that cut metal tube 100 will return from an expanded configuration to its compressed configuration when the axial compression force (which causes the radial expansion) is removed. In some embodiments, cut metal tube 100 may be made of a metal with relatively plastic properties, e.g., stainless steel, such that it will remain in its expanded configuration even after the axial compression force is removed.

The cut patterns on cut metal tube 100 may be made with a laser. In some embodiments, non-radial or off-center cuts may be made on the tubular walls of cut metal tube 100 to produce angled cuts that facilitate radial expansion of the tube. In such embodiments, application of an axial compression force results in relative radial motion of the surfaces on opposite sides of the non-radial cut. Further, in such embodiments, the adjacent sections of the tubular wall on opposite sides of the non-radial cut may form a ramp to induce the adjacent sections to overlap each other as the tube expands. The non-radial cuts may further produce adjacent surfaces that when pressed against each other in the circumferential direction induces force upon each other in the radial direction.

In exemplary embodiments, cut metal tube 100 may have a combination of radial and non-radial cuts, with regions of non-radial cuts interspersed between radial cuts. If all of the cuts in cut metal tube 100 are made radially, the friction between adjacent sections of the cuts would impede intended radial expansion when the tube is compressed axially. With non-radial cuts, the adjacent sections overlap each other when axial compression is applied, instead of pressing against each other, thereby facilitating radial expansion.

In some embodiments, the non-radial cuts may provide the expanded cut metal tube 100 with enhanced functionality, such as, providing sharp edges or angled corners which can be used as blades for cutting, grasping, rasping, scraping, or grinding. In one embodiment, non-radial cuts may produce sharp corners with an inner corner angle of less than 80°.

Generally, radial cuts in a tube are aligned with the radial plane of the tube, whereas non-radial cuts are not aligned with the radial plane of the tube. In other words, the plane of a non-radial cut forms a non-perpendicular angle with a plane tangent to the outer surface of the tubular wall at the outer edge of the cut. The angle of a non-radial cut is defined as the angle made by the plane of the non-radial cut with the radial plane that intersects the non-radial plane at the outer edge of the cut.

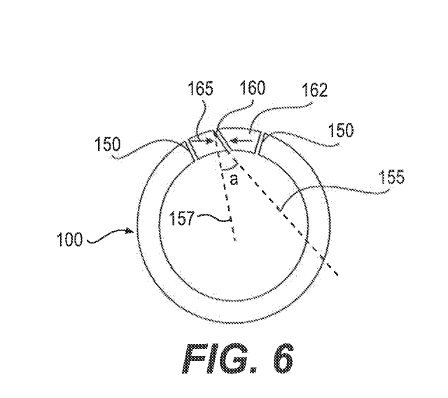

FIG. 6 shows a cross-section of an exemplary cut metal tube 100 having radial cuts 150 and a non-radial cut 160. As shown in FIG. 6, radial cuts 150 are aligned with the radius of cut metal tube 100, whereas non-radial cut 160 is not aligned with the radius of cut metal tube 100. In exemplary embodiments, the angle of a non-radial cut, i.e., the angle ‘a’ between non-radial plane 155 and radial plane 157 in FIG. 6, may be between 5° and 60°. In one such embodiment, the angle of non-radial cut 160 is 44°. Further, in some exemplary embodiments, the distance of non-radial cut plane 155 from the longitudinal axis is between 10% and 85% of the radius of cut metal tube 100.

FIG. 6 also shows the direction of forces on the two adjacent sections 162 and 165 of the tubular wall on opposite sides of non-radial cut 160. Application of axial compression force on cut metal tube 100 may result in radial motion of sections 162 and 165. Section 162 may be induced to move outwards from the center of the tube and section 165 may be induced to move inwards towards the center of the tube as a result of the non-radial cut between them.

In exemplary embodiments, cut metal tube 100 may have transition sections between the radial cut sections and the non-radial cut sections. In the transition section, the cut angle varies smoothly from the radial cut angle (0°) to the non-radial cut angle.

Some disclosed embodiments include an expandable cut metal tube covered by an elastic material, such that when expanded by applying a force along the longitudinal axis of the tube from both ends of the device, the elastic material is stretched between the bands of the cut metal tube. The elastic material may be silicone, or POLYBLEND™ (AdvanSource Biomaterials, Wilmington, Mass.), or CHRONOPRENE™ (AdvanSource Biomaterials, Wilmington, Mass.), or any similar elastic material that has appropriate elasticity and strength to stretch over the tube in its expanded configuration and return to its original dimensions when the tube returns to its compressed configuration. In exemplary embodiments, the thickness of the elastic material covering the expandable metal tube is between 10 and 250 microns.

The foregoing description has been presented for purposes of illustration. It is not exhaustive and is not limited to the precise forms or embodiments disclosed. Modifications and adaptations will be apparent to those skilled in the art from consideration of the specification and practice of the disclosed embodiment.

Moreover, while illustrative embodiments have been described herein, the disclosure includes the scope of any and all embodiments having equivalent elements, modifications, omissions, combinations (e.g., of aspects across various embodiments), adaptations and/or alterations as would be appreciated by those skilled in the art based on the present disclosure. The limitations in the claims are to be interpreted broadly based on the language employed in the claims and not limited to examples described in the present specification or during the prosecution of the application. The examples are to be construed as non-exclusive. Furthermore, the steps of the disclosed methods may be modified in any manner, including by reordering steps and/or inserting or deleting steps. It is intended, therefore, that the specification and examples be considered as illustrative only, with a true scope and spirit being indicated by the following claims and their full scope of equivalents.

1. An anchor device, comprising:
an expandable tube having a first end region and a second end region, and configured to traverse a perforation in a tissue wall of a body canal and to fit within an interior of the body canal proximate to the perforation, the expandable tube including a plurality of primary slits therein, each primary slit extending from the first end region to the second end region, the primary slits cooperating to define a plurality of bands and each primary slit including:
at least one substantially longitudinal cut portion; and
at least one substantially non-longitudinal cut portion extending from the at least one substantially longitudinal cut portion; and
wherein the primary slits are configured such that when the first end region and the second end region are compressed towards each other, the plurality of bands splay outward.

2. The anchor device of claim 1, further comprising an elastic membrane covering the expandable tube.

3. The anchor device of claim 1, wherein the at least one substantially longitudinal cut portion and the at least one substantially non-longitudinal cut portion cause each primary slit to have a stepped appearance.

4. The anchor device of claim 1, wherein the at least one substantially non-longitudinal cut portion includes two substantially non-longitudinal cut portions.

5. The anchor device of claim 1, wherein each band further includes at least one secondary slit therein, each secondary slit having a length shorter than the primary slit.

6. The anchor device of claim 1, wherein the bands are configured to splay outward in the form of petals.

7. The anchor device of claim 1, wherein the expandable tube is made of Nitinol.

8. The anchor device of claim 1, wherein the first end region and the second end region comprise cut patterns to facilitate radial expansion of the plurality of bands.

9. The anchor device of claim 1, wherein the at least one substantially non-longitudinal cut portion extends more than 150° around the expandable tube.

10. The anchor device of claim 1, wherein the at least one substantially non-longitudinal cut portion includes at least two substantially non-longitudinally cut portions, and wherein the at least two substantially non-longitudinal cut portions extend more than 150° around the expandable tube.

11. The anchor device of claim 1, wherein the expandable tube includes non-radial cuts.

12. An anchor device, comprising:
an expandable tube configured to traverse a perforation in a tissue wall of a body canal and to fit within an interior of the body canal proximate to the perforation, the expandable tube having an elongated central axis and a tubular wall configured to expand radially upon application of an axial compression force along a direction of the central axis; and
at least one non-radial cut in the tubular wall, such that application of the axial compression force results in relative radial motion of surfaces on opposite sides of the cut.

13. The anchor device of claim 12, wherein the surfaces on opposite sides of the cut form a non-perpendicular angle with a plane tangent to an outer surface of the expandable tube at a point of intersection with an outer edge of the non-radial cut.

14. The anchor device of claim 12, wherein the non-radial cut is in a plane that forms an angle of between 5° and 60° at a point of intersection with a radial plane of the expandable tube at an outer edge of the non-radial cut.

15. The anchor device of claim 12, wherein the non-radial cut is spaced from the central axis by a distance of between 10% and 85% of the radius of the expandable tube.

16. The anchor device of claim 12, wherein the non-radial cut produces at least one sharp corner for use in cutting, scraping, grinding, grasping, or rasping.

17. An anchor device, comprising:
an expandable tube having a first end region and a second end region, and configured to traverse a perforation in a tissue wall of a body canal and to fit within an interior of the body canal proximate to the perforation, the expandable tube including a plurality of primary slits therein, each primary slit extending from the first end region to the second end region, the primary slits cooperating to define a plurality of bands; and
wherein the primary slits are configured such that when the first end region and the second end region are compressed toward each other, the bands splay outward to form an overlapping petal pattern.

18. The anchor device of claim 17, wherein each band further includes at least one secondary slit therein, each secondary slit having a length shorter than the primary slits.

19. The anchor device of claim 17, wherein the primary slits extend more than 150° degrees around the expandable tube.

20. The anchor device of claim 17, wherein the primary slits comprise a combination of cuts creating a stepped appearance.

21. The anchor device of claim 17, wherein the device is configured such that application of the axial compression force to the expandable tube results in relative radial motion of the surfaces on opposite sides of the cut.

22. An anchor device, comprising:
a cut tube configured to traverse a perforation in a tissue wall of a body canal and to fit within an interior of the body canal opposite the perforation, the cut tube having a tubular wall configured to expand radially upon application of an axial compression force; and
at least one cut in a non-radial plane through the tubular wall such that adjacent surfaces on opposite sides of the cut are ramped with respect to each other, and wherein application of the axial compression force to the cut tube results in relative radial motion of the surfaces on opposite sides of the cut.

23. The anchor device of claim 22, wherein the ramped adjacent surfaces are configured to induce the adjacent sections of the tubular wall to overlap each other when pushed against each other by the deformation of the tube.

24. The anchor device of claim 22, wherein surfaces on both sides of the cut form a non-perpendicular angle with a plane tangent to the outer surface of the tube at the outer edge of the cut.

25. The anchor device of claim 22, wherein the cut in the non-radial plane is in a plane that makes an angle of between 5° and 60° with the radial plane where they meet at the outer surface of the tube.

26. The anchor device of claim 22, wherein the distance of the non-radial cut plane from a central longitudinal axis of the tube is between 10% and 85% of the radius of the tube.

27. An anchor device, comprising:
an expandable tube having a first end region and a second end region and configured to traverse a perforation in a tissue wall of a body canal and to fit within an interior of the body canal proximate the perforation, the expandable tube including a plurality of primary slits therein, each primary slit extending from the first end region to the second end region, the primary slits cooperating to define a plurality of bands, and each primary slit connected to a serpentine cut on at least one of the first end region and the second end region; and
wherein the primary slits are configured such that when the first end region and the second end region are compressed toward each other, the bands splay outward.

28. The anchor device of claim 27, wherein each serpentine cut forms an s-shape.

29. The anchor device of claim 27, wherein a serpentine cut is formed on opposing ends of each primary slit.

30. The anchor device of claim 27, wherein each primary slit has a step cut.

31. The anchor device of claim 27, wherein each primary slit includes at least one radial cut portion and at least one non-radial cut portion.

32. The anchor device of claim 27, wherein each band further includes at least one secondary slit therein, each secondary slit having a length shorter than a length of the primary slit.

33. The anchor device of claim 27, wherein the bands are configured to splay outward in the form of petals.

34. An anchor device, comprising:
an expandable tube having a first end region and a second end region and configured to traverse a perforation in a tissue wall of a body canal and to fit within an interior of the body canal proximate the perforation;
a plurality of primary slits in the expandable tube, each primary slit extending from the first end region to the second end region, the primary slits cooperating to define a plurality of bands;
at least one secondary slit within each band, the at least one secondary slit having a length shorter than a length of the primary slit.

35. The anchor device of claim 34, wherein each primary slit includes at least one radial cut portion and at least one non-radial cut portion.

36. The anchor device of claim 34, wherein each primary slit has a step-cut through a majority of a length thereof.

37. The anchor device of claim 34, wherein each primary slit has a constant pitch throughout a majority of a length thereof.

38. The anchor device of claim 34, wherein the primary slits are configured such that when the first end region and the second end region are compressed toward each other, the bands splay outward.
